(19) United States
(12) Patent Application Publication
(10) Pub. No.: US 2024/0376139 A1
(43) Pub. Date: Nov. 14, 2024

(54) MANGANESE-BASED ORGANIC FRAMEWORK AND PREPARATION METHOD AND DRUG DELIVERY METHOD

(71) Applicant: Hong Kong Centre for Cerebro-Cardiovascular Health Engineering Limited, Pak Shek Kok (HK)

(72) Inventors: Bee Luan Khoo, Pak Shek Kok (HK); Mohamed Elhousseini Hilal, Pak Shek Kok (HK)

(21) Appl. No.: 18/519,493

(22) Filed: Nov. 27, 2023

(30) Foreign Application Priority Data
May 12, 2023 (CN) 202310540938.1

(51) Int. Cl. C07F 13/00; A61K 47/24; A61K 49/00

(52) U.S. Cl. CPC C07F 13/005; A61K 47/24; A61K 49/0032

(57) ABSTRACT

This invention relates to a manganese-based organic framework and its preparation method, as well as a drug delivery method. The manganese-based organic framework is formed by coordinating metal manganese ions (Mn) with the ligand 1,1,2,2-tetracarboxyphenylethylene. The structure of the manganese-based organic framework is Mn₂(TCPE)(H₂O), where TCPE is the fluorescent probe 1,1,2,2-tetracarboxyphenylethylene. The manganese-based organic framework can serve as a high drug-loading nanocarrier, capable of loading small to medium-sized active drug molecules for the treatment of cardiovascular diseases, cancer, microbial infections, wound healing, diabetes, Alzheimer's disease, tuberculosis, etc. Additionally, it possesses label-free intrinsic fluorescence capability, which plays a crucial role in the practical experiments and interventions for detecting nanocarriers and rapidly tracking drugs.

### TECHNICAL FIELD

The present invention relates to the field of drug delivery, and in particular to a manganese-based organic framework based on manganese ions and 1,1,2,2-tetrakis(4-carboxyphenyl)ethylene, its preparation method, and drug delivery method.

### BACKGROUND

According to data from the World Health Organization (WHO), cardiovascular disease (CVD) kills nearly 18 million people every year and is one of the leading causes of death worldwide. About 22 million people die from cardiovascular and cerebrovascular diseases every year. These diseases cost more than $860 billion annually, an economic loss unparalleled by any other disease. With this in mind, researchers have been working to develop new technologies to enhance the effectiveness of existing treatments. Statins, antiplatelet drugs, anticoagulant drugs, antiarrhythmic drugs, and antihypertensive drugs are common drugs for cardiovascular disease (CVD), and the effectiveness of these drugs has been widely proven. However, after taking these drugs, patients can experience adverse effects ranging from muscle pain, fatigue and nausea to dangerous heart arrhythmias, kidney damage and bleeding.

Fortunately, drug delivery is a well-established and powerful technology that can effectively address the above shortcomings. By delivering therapeutic molecules to specific locations, it allows targeted drug delivery, thereby increasing efficacy and reducing drug-related side effects. Nanomaterials such as liposomes, polymers, quantum dots, and gold nanoparticles have been widely used to deliver therapeutic compounds to treat cardiovascular diseases. However, drug delivery is often hindered by the lack of the ability to accurately target the site of administration, which hinders the drug development process. At present, fluorescence microscopy has been widely used to accurately observe drug delivery in cardiovascular tissue systems, which can identify areas of inefficiency in drug delivery and provide some potential solutions to improve the problem. Therefore, fluorescent nanocarriers are of great significance in studying dynamic changes in drug release and monitoring adverse reactions.

However, most of the existing fluorescence monitoring technologies use commercially available fluorescent dyes (such as rhodamine, Cy5, FITC . . . ) and fix them on nanocarriers so that they emit light when excited by external radiation sources. Optical signals allow monitoring of the position and structure of nanocarriers. But fluorescent dye labelling is expensive and time-consuming, and moreover, it may compromise the stability, effectiveness, and selectivity of the nanocarriers in the process. More often, incorrect labelling or probe leakage from porous nanocarriers can lead to inaccurate or erroneous results.

### SUMMARY OF THE INVENTION

It is preferable to provide a label-free, stable fluorescent manganese-based organic framework that possesses synergistic multifunctionality. This framework is based on manganese ions and 1,1,2,2-tetracarboxyethyl-ethene (TCPE), exhibiting properties of coordination polymers with high surface area-to-volume ratio. It also possesses excellent drug loading capacity, stability, and compatibility.

It is also preferable to provide a method for preparing the aforementioned manganese-based organic framework based on manganese ions and 1,1,2,2-tetracarboxyethyl-ethene (TCPE), and to provide a drug delivery method that utilizes the aforementioned manganese-based organic framework as a delivery material.

In accordance with a first aspect of the invention, there is provided a manganese-based organic framework, wherein the structure of the manganese-based organic framework is Mn₂(TCPE)(H2O), and wherein TCPE is fluorescent probe 1,1,2,2-tetracarboxyphenylethylene.

In accordance with the first aspect, the manganese-based organic framework is of a size in a nanoscale range between 50 nm-20 μm.

In accordance with the first aspect, the manganese-based organic framework is arranged to load drugs inside the pores of the manganese-based organic framework, on the inner and outer surfaces of nanoparticles, and between aggregates.

In accordance with the first aspect, the manganese-based organic framework is arranged to be used for anticoagulants, antiplatelets, anti-inflammatory drugs, gases, anticancer drugs and other drugs.

In accordance with the first aspect, the gases include nitric oxide, hydrogen, hydrogen sulfide and carbon monoxide.

In accordance with the first aspect, the manganese-based organic framework is arranged to come into contact with a target object through patches, implants, bandages, stents, catheters, or other implantable or non-implantable devices for delivery, treatment, and/or diagnosis.

In accordance with a second aspect of the invention, there is provided a method for preparing the manganese-based organic framework in accordance with the first aspect, comprising the steps of:
1) adding a manganese precursor to a predetermined amount of solvent to obtain a manganese precursor solution;
2) diluting 1,1,2,2-tetracarboxyphenylethylene with a predetermined amount of distilled water, or adding 1,1,2,2-tetracarboxyphenylethylene to distilled water containing dimethylformamide to obtain a ligand solution;
3) deprotonating the ligand solution;
4) gradually adding the deprotonated ligand solution into the manganese precursor solution at a constant rate to obtain a mix solution containing the deprotonated ligand solution and the manganese precursor solution;
5) after stirring the mixed solution for a designated time in step 4, allowing it to settle, then precipitates at a bottom of the mixed solution is washed and centrifuged to obtain the manganese-based organic framework, and then the manganese-based organic framework is obtained and dried in a vacuum oven.

In accordance with the second aspect, in Step 3), deprotonating the ligand solution includes adding one or more bases to the ligand solution for deprotonation.

In an embodiment of the second aspect, the one or more bases may include potassium hydroxide (KOH), sodium hydroxide (NaOH), Sodium hydride (NaH), Potassium tert-butoxide (KOtBu), Lithium diisopropylamide (LDA), Sodium amide (NaNH₂), Lithium hexamethyldisilazide (LiHMDS) or any one or combination thereof.

In accordance with the second aspect, the deprotonated solution to the ligand solution is of a ratio in a range of 1:2˜1:4.

In accordance with the second aspect, a solvent content of the ligand solution does not exceed ¼, where the solvent is distilled water or distilled water containing dimethylformamide.

In accordance with the second aspect, in Step 4) the mixed solution is stirred in a temperature range of 25° C. to 140° C.

In accordance with the second aspect, in Step 4) the mixed solution is stirred for 8 hours to 5 days.

In accordance with the second aspect, in step 4) the mixed solution into multiple experimental groups for stirring according to different temperatures.

In accordance with the second aspect, in Step 5) the mixed solution is allowed to settle in the dark.

In accordance with the second aspect, in Step 5) the mixed solution is centrifuged multiple times to obtain the manganese-based organic framework.

In accordance with a third aspect of the invention, there is provided a drug delivery method, characterized by using the manganese-based organic framework as in accordance with the first aspect as a drug delivery material, or using a substance obtained by the method for preparing the manganese-based organic framework in accordance with the second aspect as a drug delivery material.

In accordance with the third aspect, the method further comprises the step of including a drug loading process and a drug release process.

In accordance with the third aspect, the drug loading process comprising the steps of: suspending a target drug in an adequate solution;
adding the drug delivery material to the adequate solution containing the target drug in a predetermined ratio;
continuously stirring a suspension containing the target drug for 1 hour to 5 days; and centrifuging the suspension multiple times until a supernatant becomes clear; In accordance with the third aspect, the drug release process comprising the steps of: suspending the drug-loaded drug delivery material in a solution;
injecting the drug-loaded drug delivery material containing solution into different dialysis membranes;
immersing the dialysis membranes into containers containing phosphate buffer solutions with different pH values; and keeping the containers at a constant speed on a shaker at a predetermined temperature.

In accordance with the third aspect, a drug loading capacity (LC %) and a drug loading efficiency (LE %) of the drug delivery material is obtained by:

LC%=(M_{T}-M_{uT})/(M_{T}-M_{uT}+M_{C})100; and

LE%=(M_{T}-M_{uT})/M_{uT}*100;
wherein M_{T}, M_{uT}, and M_{C} denote respectively a total amount of the target drug used in a formulation, an unloaded amount of the target drug, and an amount of the drug delivery material.

Compared with the existing technology, the solution of this invention has the following advantages:

1. The manganese-based organic framework based on manganese ions and 1,1,2,2,-tetrakis(4-carboxyphenyl)ethylene involved in this invention is generated through the coordination connection of metal manganese ions and TCPE ligands, providing a sufficient The expanded three-dimensional cavity network and developed porous structure enable the manganese-based organic framework to be used as a high drug loading nanocarrier, which can load small and medium-sized active drug molecules for the treatment of cardiovascular diseases, cancer, Microbial infection, wound healing, diabetes, Alzheimer's disease, tuberculosis, etc.
2. The manganese-based organic framework based on manganese ions and 1,1,2,2,-tetrakis(4-carboxyphenyl)ethylene involved in this invention has label-free and inherent fluorescence capabilities, which can detect nanocarriers and rapidly Actual experiments and interventions for tracking drugs play a vital role, and the photophysical properties of the manganese-based organic framework of the present invention are inherited by its bridging ligand TCPE, making the manganese-based organic framework of the present invention have good stability.
3. The manganese-based organic framework based on manganese ions and 1,1,2,2,-tetrakis(4-carboxyphenyl)ethylene involved in this invention exhibits pH-triggered release in slightly alkaline media, even in highly corrosive environments Drug release can also be significantly inhibited in acidic media.
4. In the preparation method of the manganese-based organic framework based on manganese ions and 1,1,2,2,-tetrakis(4-carboxyphenyl)ethylene involved in this invention, the manufacturing process has been optimized, the energy consumption in the preparation process is minimal, and the operation is Simple, aqueous conditions are used to avoid toxicity and pore contamination, eliminating any toxicity and additional costs that may arise from commonly used solvents, facilitating high-volume manufacturing.
5. The manganese-based organic framework based on manganese ions and 1,1,2,2,-tetrakis(4-carboxyphenyl)ethylene involved in this invention can encapsulate different active drug molecules and release them when stimulated, while providing monitoring And to provide a versatile imaging modality for diagnosis, the manganese-based organic framework of the present invention can induce emission in the polymerized state and be used for photodynamic or thermodynamic treatment of cardiovascular diseases and other diseases.

Additional aspects and advantages of the invention will be set forth in part in the description which follows, and will be obvious from the description, or may be learned by practice of the invention.

### BRIEF DESCRIPTION OF THE DRAWINGS

The above-mentioned and/or additional aspects and advantages of the present invention will become apparent and easy to understand from the following description of embodiments in conjunction with the accompanying drawings, in which:

FIG. 1 shows X-ray diffraction patterns collected from 7° to 80° for three synthesized metal-organic frameworks (CCH-1, 2, and 3).

FIG. 2 displays the Fourier-transform infrared spectroscopy of the metal-organic framework (CCH-2 and TCPE) in the range of 400 to 3600 cm-1.

FIG. 3 presents scanning electron microscope images of CCH-1 (scale bar=1 m).

FIG. 4 exhibits the ultraviolet-visible absorption spectra of CCH-1, CCH-2, and CCH-3.

FIG. 5 shows the photoluminescence spectra of CCH-2 and TCPE, with an emission wavelength of 320 nm.

FIG. 6 displays microscopic imaging of CCH-3 in DMEM under bright-field (A), 550 nm excitation (B), 385 nm excitation (C), and 475 nm excitation (D).

FIG. 7 represents the ultraviolet-visible spectroscopy analysis of blood compatibility for CCH.

FIG. 8 illustrates the cell viability assay for lung fibroblast cells.

FIG. 9 depicts the cumulative release curve of tetracycline from CCH-2 nanocarriers at pH 7.4 and 5.5 in PBS.

FIG. 10 presents the fitting model for the cumulative tetracycline release data.

### DETAILED DESCRIPTION OF THE PREFERRED EMBODIMENTS

The embodiments of the present invention are described in detail below. Examples of the embodiments are shown in the accompanying drawings, wherein the same or similar reference numerals throughout represent the same or similar elements or elements with the same or similar functions. The embodiments described below with reference to the drawings are exemplary and are only used to explain the present invention and cannot be construed as limiting the present invention.

Aiming at the problem of medication for acute cardiovascular diseases, this disclosure provides a manganese-based organic framework based on manganese ions and 1,1,2,2,-tetrakis(4-carboxyphenyl)ethylene. And the manganese-based organic framework of 1,1,2,2,-tetrakis(4-carboxyphenyl)ethylene is used as the basic nanocarrier, which can simultaneously provide functions such as efficient drug loading, controlled release and specific range of photoluminescence.

Preferably, the chemical structural formula of the manganese-based organic framework of the present invention based on manganese ions and 1,1,2,2-tetrakis(4-carboxyphenyl)ethylene is Mn₂(TCPE)(H₂O), wherein the TCPE is the bridging ligand which is specifically the fluorescent probe 1,1,2,2,-tetrakis(4-carboxyphenyl)ethylene. The manganese-based organic framework of the present invention can be used as a label-free fluorescent MOF nanocarrier, has nanoscale small size, good biocompatibility and controllable drug release capabilities, and can provide efficient and simultaneous combined intervention to make drugs being tracked in real time within the body, while enabling powerful, controllable drug delivery.

The above-mentioned manganese-based organic framework based on manganese ions and 1,1,2,2,-tetrakis(4-carboxyphenyl)ethylene can be specifically obtained through the following preparation method, which preparation method includes the following steps:
1) Add the manganese precursor to an appropriate amount of solvent. In one preferred embodiment, manganese chloride tetrahydrate is added to deionized water and stirred until the metal salt is obviously dissolved.
2) Use 1,1,2,2-tetrakis(4-carboxyphenyl)ethylene (i.e. fluorescent probe 1,1,2,2-tetrakis(4-carboxyphenyl)ethylene, hereinafter referred to as “TCPE”). Add and dilute in an appropriate amount of distilled water, or add TCPE to distilled water with dimethylformamide added to obtain a ligand solution, that is, the ligand solution uses distilled water or distilled water with dimethylformamide added as TCPE. Preferably, the solvent content in the ligand solution does not exceed ¼ of the total solution.
3) Deprotonate the ligand solution. Deprotonation is the process of removing protons (H) from molecules to generate their conjugate bases. In this embodiment, the solution is deprotonated by adding an appropriate amount of potassium hydroxide or sodium hydroxide, although other related, similar or suitable bases may also be used, including, without limitations, Sodium hydride (NaH), Potassium tert-butoxide (KOtBu), Lithium diisopropylamide (LDA), Sodium amide (NaNH₂), Lithium hexamethyldisilazide (LiHMDS) or any one or combination thereof. Preferably, the pH of the ligand solution may be adjusted to achieve deprotonation. Further preferably, the ratio of the alkaline solution added for deprotonation to the ligand solution ranges from 1:2 to 1:4.
4) Add the deprotonated ligand solution in step 3) to the manganese precursor solution in a controlled manner, and different temperature values within the set temperature range may be selected to stir the mixed solution to obtain different compositions. The deprotonated ligand solution is gradually added to the prepared manganese precursor solution in step 1) in a controlled manner, e.g. at a constant rate. Preferably, the temperature range may be 25° C. to 60° C. in this step, although it may be between 25° C. to 140° C. In the experiments performed by the inventors, three groups of mixed solutions were obtained by dividing the mixed solution and were stirred at room temperature (25° C.), 40° C. and 60° C. respectively.
5) After the mixed solution in step 4) is stirred for a set time, let it stand in the dark, wash and centrifuge the lower precipitate multiple times to obtain the manganese-based organic framework compound, and manganese-based organic framework compound may be obtained. The manganese-based organic framework may be further dried in a vacuum oven.

In one example embodiment, the stirring time is 24 hours to 5 days, although it can be as short as one hour. Then, the stirred mixed solution is placed at room temperature in the dark and left to stand. The supernatant is taken out and stored for subsequent experimental analysis. The precipitate is further processed by multiple cleaning and centrifugation are dried under vacuum at 60-80° C. for 12-16 hours to obtain the manganese-based organic framework in accordance with an embodiment of the present invention.

In one example embodiment, the manganese-based organic framework CCH is synthesized by mixing TCPE ligands and manganese ions in an aqueous solution, and the carboxylic acid groups on the TCPE ligands are deprotonated by alkaline addition. In addition, three experimental groups, i.e. CCH-1, CCH-2, and CCH-3, were prepared according to different stirring temperatures. The size of the CCH prepared is a nanoscale size between 200-10 μm. In the following disclosure, the CCH prepared will be measured from the structural and morphological characteristics of CCH, the photophysical properties of CCH, the biocompatibility of CCH, and drug loading and release.

### Structural and Morphological Characterization of CCH

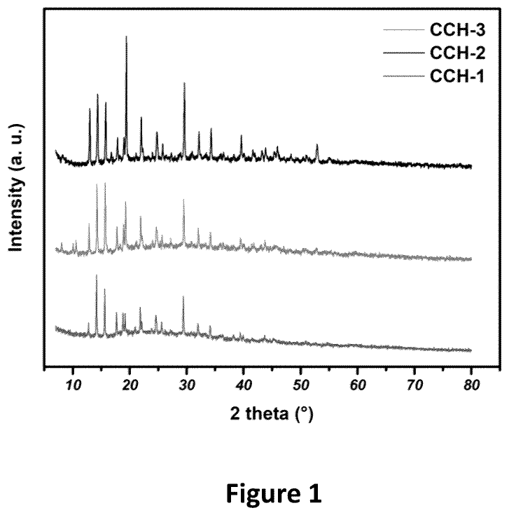

The inventors used X-ray diffraction (XRD) to analyze the structural and morphological characteristics of the obtained manganese-based organic framework CCH. With reference to FIG. 1, there is shown three synthetic manganese-based organic frameworks CCH-1, CCH-2, and CCH-3. X-ray diffraction patterns collected from frames 7° to 80° with a Bruker D2 PHASER X-ray diffractometer in theta-theta configuration with a scan step of 0.03 s and a coupling tension/current of 15 kV/40 mA was applied to the copper anode, X-ray diffraction in the range 7° to 80° was obtained. As can be seen from FIG. 1, the three manganese-based organic frameworks CCH-1, CCH-2, and CCH-3 are at 20=8.07°, 10.07°, 10.62°, 12.88°, 14.27°, 15.72°, 16.56°, 17.77°, 18.90°, 19.32°, 21.14°, 21.95°, 24.69°, 25.71°, 27.24°, 29.50°, 32.07° and 34.23° all show X-ray diffraction. The above-mentioned well-decomposed dense peaks indicate that the synthesis of the CCH powder belongs to highly crystalline metal organic framework compounds (MOFs). In addition, using Scherrer's formula to estimate, D=(0.94*λ)/(β*Cos(θ)), where D is the average grain size, β is the line width (radians), θ is the Bragg angle, and λ is 0.154056 nanometers. At the X-ray wavelength, CCH-2 shows a smaller average grain size, only about 39 nm. The smaller the crystal size, the more it indicates that the nanomaterial has high porosity and surface area, and the gaps between the grains produce abundant micropores, which give it a three-dimensional expanded porous structure and the ability to carry high drug loading.

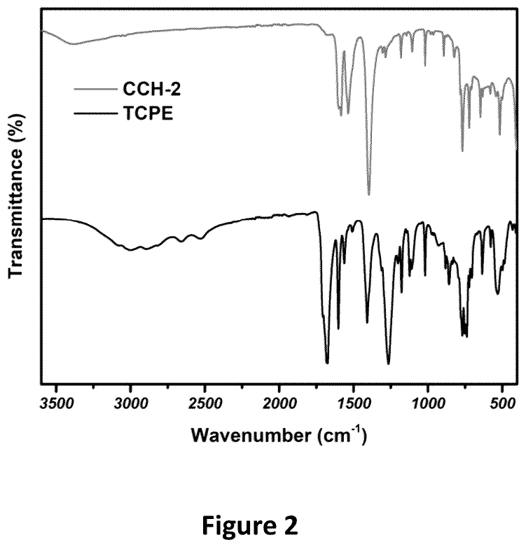

In order to further verify that the compound prepared by the preparation method of this invention is a manganese-based organic framework, the Fourier transform infrared (FTIR) and attenuated total reflection (ATR) spectra of MOFs were tested on a Thermo Scientific Nicolet iS5 spectrometer, and the spectral resolution is 4 cm⁻¹. See FIG. 2 for details.

FIG. 2 shows the Fourier transform infrared spectrum of metal organic frameworks (CCH-2 and TCPE) in the range of 400 to 3600 cm⁻¹. In the powder state, its absorption peaks are located at 3395 cm⁻¹, 1664 cm⁻¹, 1586 cm⁻¹ and 1536 cm⁻¹, 1398 cm⁻¹, 1181 cm⁻¹, 1104 cm⁻¹, 1018 cm⁻¹ and 891 cm⁻¹, 770 cm⁻¹, 720 cm⁻¹, 645 cm⁻¹ and 514 cm⁻¹, the absorption peaks of CCH-2 and TCPE in the wavelength region of 600-1700 nm characterize the typical vibration bands of carboxyl (—COO—) and carbonyl (—C═O), The absorption peaks at 1398 cm⁻¹ and 1664 cm⁻¹ come from —C═C— and 1,1,2,2,-tetrakis(4-carboxyphenyl)ethylene. Normally, pure TCPE will show the stretching frequency of —CO at 1682 cm⁻¹. However, the curve of CCH-2 does not show this peak, confirming the existence of free ligand molecules, while—CO stretching The transition to 1598 cm⁻¹ confirms the formation of the manganese-based organic framework CCH-2. The peaks at 1664 cm−1 and 1018 cm−1 are assigned to the C—H bending vibration of the benzene ring and the vibration of the carboxylate group, respectively. In contrast, the low band around 514 cm¹ can be attributed to the oxygen from the carboxyl group and the metal. The metal-oxygen bond between them further confirmed the successful formation of manganese-based organic framework CCH-2.

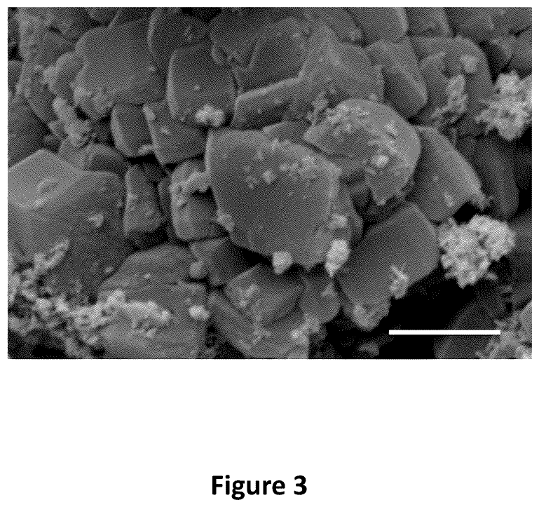

In addition, a scanning electron microscope FEI Quanta 250 Environmenta can also be used to observe the shape and size of the synthesized manganese-based organic framework. It can be seen from the scanning electron microscope micrograph of CCH-1 in FIG. 3 (scale bar=1 um) that at room temperature. The CCH-1 synthesized by using deionized water has a clear crystallized polyhedral morphology, and its size is between 400 nm and 800 nm, which is within the size range of the CCH prepared in this invention.

### Photophysical Properties of CCH

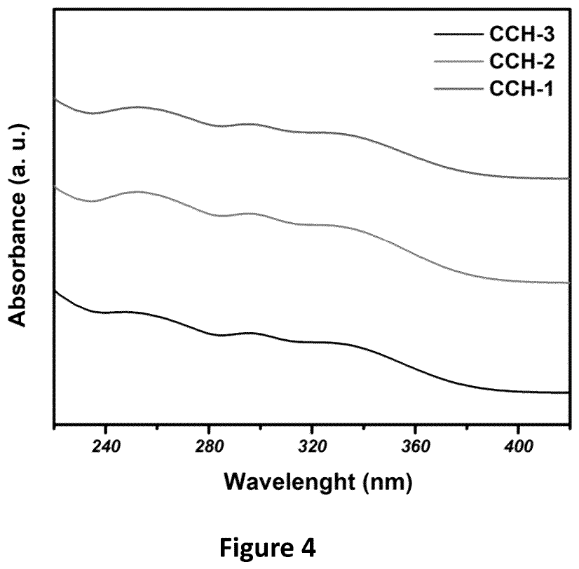

Regarding the photophysical properties of the synthesized manganese-based organic framework CCH, the inventors studied its UV absorbance and photoluminescence through UV-visible light and fluorescence spectroscopy, where the UV-visible light absorption spectrum was measured on an Agilent CARY 3500 multi-chamber UV-visible spectrophotometer. Measured on a meter. From the UV-visible absorption spectra of CCH-1, CCH-2, and CCH-3 in FIG. 4, it can be seen that the absorption range of CCH extends from 200 nm to 400 nm along the UV spectrum, and the metal-organic framework exhibits at 295 nm and 334 nm. An absorption band, while there is a maximum absorption peak at 251 mm, these absorption wavelengths are inherited from TCPE, which further confirms the successful synthesis of CCH organometallic compounds and the molecular structure of the coordination ligands has not changed.

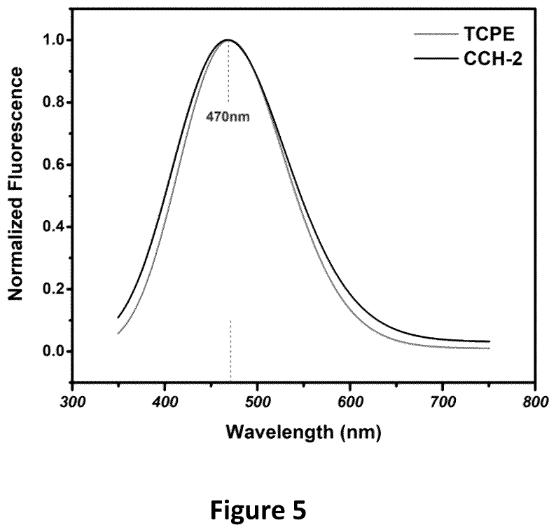

The inventors also evaluated the photoluminescence of CCH-2 and TCPE. The steady-state photoluminescence (PL) spectrum is measured on a PerkinElmer spectrofluorometer LS55. The measurement results can be seen in FIG. 5. FIG. 5 shows CCH-2 and TCPE. Photoluminescence spectrum of TCPE. As can be seen from FIG. 5, CCH-2 exhibits photoluminescence in the range of 350 nm to 750 nm, and reaches a maximum value at 460 nm, which has the same emission wavelength as the TCPE used in this invention. The manganese-based metal framework inherits the photoluminescence capabilities of its bridging ligands.

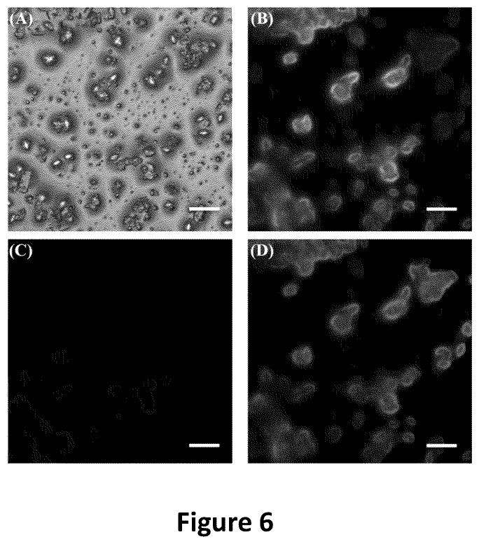

In addition, the TCPE used in this invention is an active molecule with aggregation-induced emission (AIE). If the radiation-free intramolecular movement is restricted by aggregation, it will undergo strong luminescence relaxation after irradiation. Subsequently, CCH constrains the TCPE ligands in the crystal lattice, thereby inhibiting the dynamic rotation of their benzene rings in the solid state, so the CCH grains are endowed with intrinsic high fluorescence. FIG. 6 (A), (B), (C), and (D) respectively show that the manganese-based organic framework CCH is in bright field, excited by an excitation wavelength of 550 nm, an excitation wavelength of 385 nm, and an excitation wavelength of 475 nm. Clearly visible aggregates can be seen in DMEM medium.

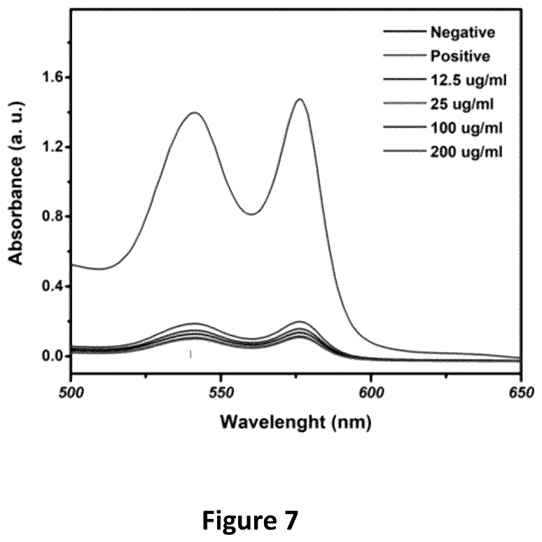

Biocompatibility of CCH In order to verify the biocompatibility of the manganese-based organic framework CCH of the present invention, a hemolysis experiment was conducted on CCH. Specifically, rat blood diluted 20 times with 0.067M phosphate buffer (pH7.4) was used, respectively, with CCH and phosphate. Salt buffer (negative control) or deionized water (positive control) were incubated for 6 hours at 150 rpm and 37° C. Hemolysis was subsequently tested as the CCH concentration increased by taking the UV-visible absorbance of the supernatant at 541 nm. The percentage of hemolysis is calculated as follows: HI=(Aₛₐₘₚₗₑ−A_{neg}/Aₚₒₛ−A_{neg})*100, where HI is the hemolysis index, Aₛₐₘₚₗₑ is the absorbance of CCH at a specific concentration, A_{neg} is the absorbance of the negative control, and Apos is the absorbance of the positive control. FIG. 7 is the UV-Vis spectrum of the blood compatibility analysis of CCH. It can be seen that the UV-Vis spectrum of all concentrations is close to the negative control and significantly lower than the positive control spectrum, which means that released free hemoglobin in the blood samples was significantly lower, as their characteristic absorption at 541 nm is very weak, which demonstrates the hemocompatibility of the CCH of the present invention. Furthermore, the hemolysis index was calculated and showed a value of 4.8% for the maximum concentration of CCH (200 μg/ml).

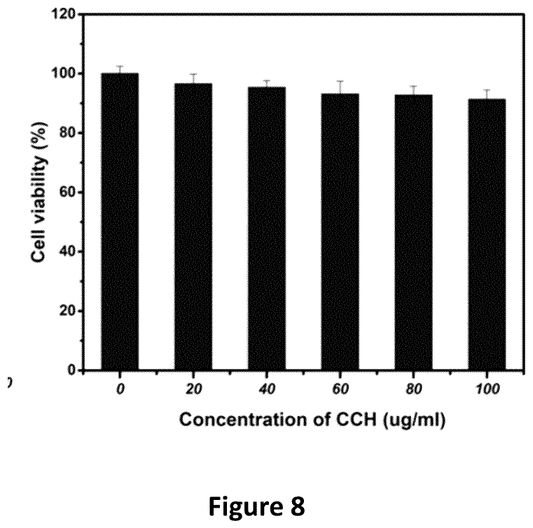

In addition, the in vitro cytotoxicity of CCH was studied by detecting the survival rate of lung fibroblasts cultured with five different concentrations (20, 40, 60, 80 and 100 μg/ml) of CCH-2 for 24 hours. ability. Cell viability was assessed by fluorescent staining. The nuclear dyes Hoechst (Invitrogen, #H1399, USA) and Propidium Iodide were diluted 1:1000 in an appropriate amount of PBS and added to lung fibroblasts after incubation at 37° C. for 20 min and 5 min respectively. Cells were then washed three times with PBS. The lung fibroblasts of this invention were cultured in DMEM (Gibco, USA) supplemented with 10% (v/v) fetal calf serum (Gibco, USA) and cultured in a humidified environment at 37° C. and 5% carbon dioxide. Lung fibroblasts were then cultured in sterile T25 flasks (Jet BIOFIL, China) and passaged after reaching 80% cell maturity. The growth medium was replaced every 48-72 h, and cells were seeded on 96 or 6-well plates (BIOFIL, China) for experiments. The cells were then imaged by fluorescence microscopy, and the images were analyzed using ImageJ software. FIG. 8 shows the cell viability detection of lung fibroblasts by CCH. The data is the average of three experiments. It can be seen that even at a higher concentration (100 μg/ml), CCH-2 No apparent toxicity was demonstrated. Therefore, it can be concluded that CCH nanocarriers have excellent biocompatibility and are ideal for biological applications.

### Drug Loading and Release

The manganese-based organic framework of the present invention has a high surface area and volume ratio, and can provide label-free inherent fluorescence capabilities to achieve efficient and synchronized combined intervention, so that drugs can be tracked in real time in the body, thereby achieving powerful and reliable drug delivery capabilities. Preferably, the present invention also relates to a drug delivery method that adopts the manganese-based organic framework of the present invention based on manganese ions and 1,1,2,2,-tetrakis(4-carboxyphenyl)ethylene as the drug delivery material.

Specifically, the drug delivery method includes a drug loading process and a drug release process, wherein the drug loading process includes the following steps: suspending the target drug in an aqueous solution, and adding the drug delivery material in a set ratio to the target drug. into an aqueous solution of the target drug, the resulting suspension was continuously stirred for 48 hours, and the suspension was centrifuged multiple times until the supernatant became clear. The drug release process includes the following steps: suspend the drug delivery material loaded with the target drug in an aqueous solution, inject it into different dialyzers with a pipette, and immerse the dialyzers into phosphate buffer saline solutions containing different pH values. in a container, and then maintain the container in a shaker at a constant speed at a preset temperature.

This example demonstrates the loading and release of a drug model on CCH-2 nanocarriers, and uses tetracycline as the target drug for loading.

First, the drug loading process is performed by suspending the tetracycline drug (2 mg) in 1 ml of aqueous solution. CCH-2 is added to the container at a mass ratio of 2:1 to the drug. The resulting suspension is continuously stirred for 48 hours. The suspension is passed through multiple Centrifuge until the supernatant becomes clear and no longer has the yellow color of the drug. CCH (Tetracyline@CCH-2) loaded with tetracycline drugs was recovered and stored for subsequent release experiments, and the supernatant was further analyzed using UV-visible light to obtain loading indicators.

Drug loading capacity (LC %) and loading efficiency (LE %) can be calculated by the following formula:

LC%=(M_{T}−M_{uT})/(M_{T}−M_{uT}+M_{C})*100; and

LE%=(M_{T}−M_{uT})/M_{uT}*100.

Among them, M_{T}, M_{uT} and M_{C} are the total amount of tetracycline used in the formula, the amount of unloaded tetracycline and the amount of CCH-2, respectively.

Calculated based on the above formula, LC %=14.46%, which has a high drug loading capacity, which is equivalent to loading 0.16 mg of drug per mg of CCH-2, and under the current loading experimental conditions, the loading efficiency reaches LE %=8.45%, and according to the calculated formula, the drug loading of CCH can be optimized from the ratio of CCH-2 to drug model, drug loading time and concentration of drug model. In addition, it should be noted that the drug models of this invention include but are not limited to coagulants (tPA, uKA, Streptokinase, Streptase, Activase, etc.), anticoagulants (Dabigatran, Apixaban, Rivaroxaban, Heparin, Warfarin, etc.), antiplatelets (Aspirin, cilostazol, atopasa, tirofiban, etc.), anti-inflammatory drugs, gases (NO, H₂, H₂S, CO, etc.), as well as anti-cancer drugs (doxorubicin, etc.) and other drugs.

The drug release in this invention is to suspend the loaded Tetracyline@CCH-2 in 1 ml of water, use a pipette to inject into two different dialyzers, and then immerse the two dialyzers in 20 ml of water with different pH values (7.4, 5.5) into a container of PBS buffer, and keep both containers in a shaker at 37° C. at a speed of 100 rpm. Release profiles can be constructed by sampling the supernatant over a specific time frame and replacing the same volume of supernatant with fresh release medium. All aspirated supernatants were saved for further analysis using UV-visible light to determine the cumulative concentration of released tetracycline as follows: Cumulative drug release=Ct*v, where Ct is the tetracycline measurement at t at the time of sampling concentration, v is the volume of the derivatized sample.

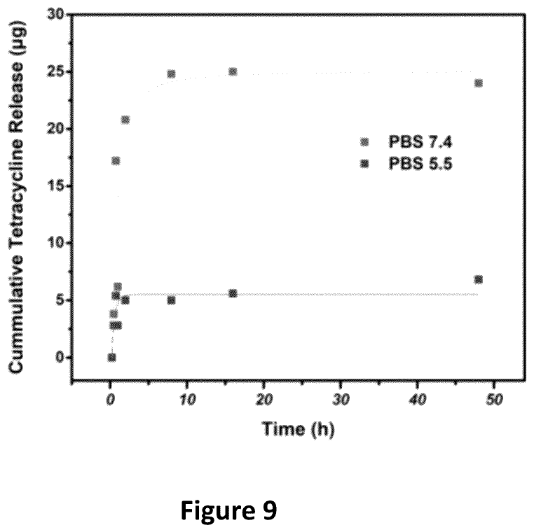

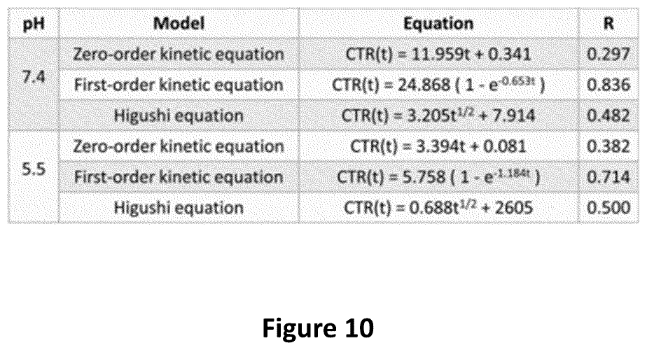

Embodiments of the present invention were evaluated by the dialysis bag method, with continuous sampling at increasingly longer time intervals, as shown in FIGS. 9 and 10. FIG. 9 shows the CCH-2 nanocarriers in PBS 7.4 and 5.5 pH. Cumulative release curve of tetracycline. FIG. 10 is a fitting model diagram of the cumulative tetracycline release data of CCH-2 nanocarrier. Tetracyline@CCH-2 showed quite different release rates under the two pH conditions, with the release rate at pH7.4 being faster than that at pH5.5 within the 48 hours of testing. And after the first 20 hours, the cumulative release of tetracycline reached 25 μg at pH 7.4 and 5.75 μg at pH 5.5. This expected slower release rate at low pH is due to the inherent fragility of the manganese ion, which allows it to dissociate from its coordination bond with the carboxylate ion and return. The release data points of Tetracyline@CCH-2 are most consistent with the first-order kinetic release equation, indicating that the drug-loaded particles have good sustained release effect, which may be related to the fact that the load in the pores of CCH-2 nanoparticles is more than the load on the outer surface.

In summary, this invention provides a label-free stable fluorescent manganese-based organic framework nanoparticle (i.e. CCH) through the coordination connection of metal manganese ions and TCPE ligands, providing a fully expanded three-dimensional cavity network and developed porous structure. Since CCH has a nanoscale size, it can load drugs within the pores, the inner and outer surfaces of nanoparticles, and between aggregates. CCH has good drug loading capacity and has good stability and biocompatibility, by loading small and medium-sized active drug molecules, it can play a certain therapeutic role in the fields of cardiovascular disease, cancer, microbial infection, wound healing, diabetes, Alzheimer's disease, tuberculosis and other diseases. In specific use, it can be used according to clinical Prescription delivery methods include, but are not limited to, intravenous, oral, pulmonary, inhaled spray, and transdermal.

CCH nanocarriers are also able to provide label-free, intrinsic fluorescence capabilities with strong emission in the visible range, marking a key feature for practical experimental and interventional monitoring of nanocarriers and rapid tracking of drugs.

CCH nanocarriers exhibit pH-triggered release in slightly alkaline media and can significantly inhibit drug release even in highly corrosive acidic media. Therefore, CCH is a reasonable candidate material as a nanocarrier for applications in microenvironments with neutral to alkaline pH, or as a fluorescent protective layer for cargoes with low pH.

The method for preparing CCH in this invention has been optimized. The preparation process consumes minimal energy and is simple to operate. At the same time, it uses aqueous solution conditions to avoid toxicity and pore contamination, eliminates any toxicity and additional costs that may be incurred by commonly used solvents, and successfully achieves large-scale manufacturing. And CCH can be produced in different forms through various common preparation methods, including but not limited to powder, colloid, film, coating, etc. Therefore, CCH has the ability to be applied, cast, or coated on a wide range of devices, including but not limited to drug delivery systems. This feature gives the final system special thermal diagnostic versatility. The application is drug-loaded or unloaded. CCH can be in contact with the target through, but not limited to, patches, implants, bandages, stents, catheters or other implantable or non-implantable devices to achieve the purpose of delivery, treatment and/or diagnosis.

The above are only some of the embodiments of the present invention. It should be noted that those of ordinary skill in the technical field may also make several improvements and modifications without departing from the principles of the present invention. These improvements and modifications may also be made and should be regarded as within the scope of protection of this invention.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

1. A manganese-based organic framework, wherein the structure of the manganese-based organic framework is Mn₂(TCPE)(H₂O), and wherein TCPE is fluorescent probe 1,1,2,2-tetracarboxyphenylethylene.

2. The manganese-based organic framework according to claim 1, wherein the manganese-based organic framework is of a size in a nanoscale range between 50 nm-20 μm.

3. The manganese-based organic framework according to claim 1, wherein the manganese-based organic framework is arranged to load drugs inside the pores of the manganese-based organic framework, on the inner and outer surfaces of nanoparticles, and between aggregates.

4. The manganese-based organic framework according to claim 3, wherein the manganese-based organic framework is arranged to be used for loading anticoagulants, antiplatelets, anti-inflammatory drugs, gases, anticancer drugs and other drugs.

5. The manganese-based organic framework according to claim 4, wherein the gases include nitric oxide, hydrogen, hydrogen sulfide and carbon monoxide.

6. The manganese-based organic framework according to claim 1, wherein the manganese-based organic framework is arranged to come in contact with a target object through patches, implants, bandages, stents, catheters, or other implantable or non-implantable devices for delivery, treatment, and/or diagnosis.

7. A method for preparing the manganese-based organic framework in accordance with claim 1, comprising the steps of:
1) adding a manganese precursor to a predetermined amount of solvent to obtain a manganese precursor solution;
2) diluting 1,1,2,2-tetracarboxyphenylethylene with a predetermined amount of distilled water, or adding 1,1,2,2-tetracarboxyphenylethylene to distilled water containing dimethylformamide to obtain a ligand solution;
3) deprotonating the ligand solution;
4) gradually adding the deprotonated ligand solution into the manganese precursor solution at a constant rate to obtain a mix solution containing the deprotonated ligand solution and the manganese precursor solution;
5) after stirring the mixed solution for a designated time in step 4, allowing it to settle, then precipitates at a bottom of the mixed solution is washed and centrifuged to obtain the manganese-based organic framework, and then the manganese-based organic framework is obtained and dried in a vacuum oven.

8. The method for preparing the manganese-based organic framework according to claim 7, wherein in Step 3), deprotonating the ligand solution includes adding one or more bases to the ligand solution for deprotonation.

9. The method for preparing the manganese-based organic framework according to claim 8, wherein the deprotonated solution to the ligand solution is of a ratio in a range of 1:2-1:4.

10. The method for preparing the manganese-based organic framework according to claim 7, wherein a solvent content of the ligand solution does not exceed ¼, where the solvent is distilled water or distilled water containing dimethylformamide.

11. The method for preparing the manganese-based organic framework according to claim 7, wherein in Step 4) the mixed solution is stirred in a temperature range of 25° C. to 140° C.

12. The method for preparing the manganese-based organic framework according to claim 7, wherein in Step 4) the mixed solution is stirred for 8 hours to 5 days.

13. The method for preparing the manganese-based organic framework according to claim 11, wherein in step 4) the mixed solution into multiple experimental groups for stirring according to different temperatures.

14. The method for preparing the manganese-based organic framework according to claim 7, wherein in Step 5) the mixed solution is allowed to settle in the dark.

15. The method for preparing the manganese-based organic framework according to claim 7, wherein in Step 5) the mixed solution is centrifuged multiple times to obtain the manganese-based organic framework.

16. A drug delivery method, characterized by using a manganese-based organic framework of Mn2(TCPE)(H2O), wherein TCPE is fluorescent probe 1,1,2,2-tetracarboxyphenylethylene, as a drug delivery material, or using a substance obtained by a method for preparing the manganese-based organic framework comprising the steps of: 1) adding a manganese precursor to a predetermined amount of solvent to obtain a manganese precursor solution; 2) diluting 1,1,2,2-tetracarboxyphenylethylene with a predetermined amount of distilled water, or adding 1,1,2,2-tetracarboxyphenylethylene to distilled water containing dimethylformamide to obtain a ligand solution; 3) deprotonating the ligand solution; 4) gradually adding the deprotonated ligand solution into the manganese precursor solution at a constant rate to obtain a mix solution containing the deprotonated ligand solution and the manganese precursor solution; 5) after stirring the mixed solution for a designated time in step 4, allowing it to settle, then precipitates at a bottom of the mixed solution is washed and centrifuged to obtain the manganese-based organic framework, and then the manganese-based organic framework is obtained and dried in a vacuum oven, as a drug delivery material.

17. The drug delivery method according to claim 16, further comprising the step of including a drug loading process and a drug release process.

18. The drug delivery method according to claim 17, wherein the drug loading process comprising the steps of:
suspending a target drug in an adequate solution;
adding the drug delivery material to the solution containing the target drug in a predetermined ratio;
continuously stirring a suspension containing the target drug for 1 hour to 5 days; and
centrifuging the suspension multiple times until a supernatant becomes clear.

19. The drug delivery method according to claim 17, wherein the drug release process comprising the steps of:
suspending the drug-loaded drug delivery material in an adequate solution;
injecting the drug-loaded drug delivery material containing solution into different dialysis membranes;
immersing the dialysis membranes into containers containing phosphate buffer solutions with different pH values; and
keeping the containers at a constant speed on a shaker at a predetermined temperature.

20. The drug delivery method according to claim 16, wherein a drug loading capacity (LC %) and a drug loading efficiency (LE %) of the drug delivery material is obtained by:
LC%=(M_{T}−M_{uT})/(M_{T}−M_{uT}+M_{C})100; and
LE%=(M_{T}−M_{uT})/M_{uT}*100;
wherein M_{T}, M_{uT}, and M_{C} denote respectively a total amount of the target drug used in a formulation, an unloaded amount of the target drug, and an amount of the drug delivery material.
